(19) United States
(12) Patent Application Publication
(10) Pub. No.: US 2014/0147391 A1
(43) Pub. Date: May 29, 2014

(54) BIOPROBE BASED ON SINGLE-PHASE UPCONVERSION NANOPARTICLES (UCNPs) FOR MULTI-MODAL BIOIMAGING

(71) Applicant: THE HONG KONG POLYTECHNIC UNIVERSITY, Hong Kong (HK)

(72) Inventors: Jianhua HAO, Hong Kong (HK); Songjun ZENG, Hong Kong (HK)

(73) Assignee: THE HONG KONG POLYTECHNIC UNIVERSITY, Hong Kong (HK)

(21) Appl. No.: 13/688,176

(22) Filed: Nov. 28, 2012

(51) Int. Cl. A61K 49/04; A61K 49/18; A61K 49/00

(52) U.S. Cl. CPC A61K 49/04; A61K 49/0019; A61K 49/1824; Y10S 977/773; Y10S 977/928; Y10S 977/93; B82Y 15/00
USPC 424/9.323; 435/29; 424/9.6; 424/9.32; 424/9.41; 424/9.411; 977/773; 977/928; 977/930; 977/927; 977/896

(57) ABSTRACT

A bioprobe based on surface-modified single-phase BaGdF₅:Yb/Er upconversion nanoparticles (UCNPs) for multi-modal bioimaging of fluorescent, magnetic resonance imaging (MRI) and computed X-ray tomography (CT) is disclosed herein. The modified UCNPs of the present invention are synthesized by a facile one-pot hydrothermal method with simultaneous surface modification of the nanoparticles. The surface-modified UCNPs of the present invention are useful in a variety of biomedical application fields due to their advantages in in vitro and in vivo multi-modal bioimaging such as small particle size up to 15 nm, substantially free of autofluorescence, low cytotoxicity, capable of being excited at near-infrared (NIR) wavelength, ability to deep cell penetration, long-lasting signal and long circulation time in vivo, different X-ray absorption coefficients at different photon energy levels between Ba and Gd, large magnetic moment, etc.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### CROSS-REFERENCE TO RELATED APPLICATIONS

There are no related patent applications

### FIELD OF THE INVENTION

The present invention relates to a bioprobe based on single-phase BaGdF₅:Yb/Er upconversion nanoparticles (UCNPs) for multi-modal bioimaging. In particular, the surface of said single-phase BaGdF₅:Yb/Er UCNPs is modified by different compounds including amino group and polyethylene glycol (PEG) moiety to become a water soluble and non-hydrophobic upconversion nanoparticles for multi-model bioimaging. The present invention also relates to methods of using said modified BaGdF₅:Yb/Er UCNPs as a bioprobe for multi-modal bioimaging of upconversion fluorescence, magnetic resonance imaging (MRI), and computed X-ray tomography (CT) imaging.

### TECHNICAL BACKGROUND

In recent years, bioimaging study has attracted much attention due to its ability to visualize and understand many functions in various biosystems ranging from specific molecules to tissues. Bioimaging techniques such as fluorescent imaging [1], computed X-ray tomography (CT) [2], and magnetic resonance imaging (MRI) [3] have played important roles in the area of bioimaging. Fluorescent imaging has been the most widely used technique among the three in biomedical imaging study. Upconversion nanoparticles (UCNPs) are emerged as a new generation of fluorescent probes for bioimaging, owing to their unique upconversion (UC) property utilizing low-energy near-infrared (NIR) light instead of high-energy ultra-violet (UV) light as an exaction source via a two- or multi-photon and/or energy transfer process [4-6]. Compared with conventional biomarkers, UCNPs possess many advantages, including reduced autofluorescence, deep tissue penetration, large anti-Stokes shifts, excellent photostability, NIR to NIR emission, and low toxicity [7-8]. Host material of UCNPs play an important role in achieving efficient UC luminescence. Among various types of investigated UC hosts, fluorides (MLnF, M=Ba, Li, Na, or K) are considered as the most promising host lattice for UC luminescence since they normally have lower phonon energy, leading to the decrease in non-radiative relaxation probability and subsequent increase in the luminescence efficiency. Much effort has focused on developing Ln³⁺ doped NaLnF₄ UCNPs. Up to now, NaLnF₄: Yb,Er/Tm UCNPs have already been extensively studied for the detection of DNA, avidin, and the fluorescent bioimaging of cells and tissues in-vitro and in-vivo [9-11]. Since the size range of the targeted biomolecules in cells and tissues is usually from several to few tens nanometers, an ideal fluorescent label should be relatively small in size accordingly, which would be compatible with the targeted biomolecules. However, the size of the reported UCNPs (20-60 nm) is not optimal for the use as bioimaging probes. It is known that the UC emission for hexagonal-phase in NaLnF₄ host is much higher than that for cubic-phase. Unfortunately, the completion of phase transition generally results in the significant particle aggregation or morphology change. Therefore, it has been challenging to prepare small NaLnF₄ nanoparticles (e.g., 10 nm) with hexagonal phase structure that can emit intense emission, although ultra-small size hexagonal NaLnF₄ NPs are recently obtained by thermal decomposition through Gd³⁺ doping [12], and refluxing process followed by hydrothermal treatment [8]. Additionally, most of the uniform hexagonal NaLnF₄ NPs are generally synthesized by using co-thermolysis in non-hydrolytic solvents or liquid solid-solution (LSS) process, which may result in hydrophobic nanoparticles [6]. Obviously, subsequent further surface modification on the hydrophobic nanoparticles is necessary for fluorescent bioimaging application. Therefore, it is of great significance to find some new UCNPs beyond NaLnF₄ host through a simple one-step route and therefore synthesize UCNPs with well-defined monodispersity, water-solubility, biocompatibility, particularly optimal size (e.g., 10 nm) suitable for bioprobe.

It is noted that the bulk BaYF₅:Yb/Er can present much brighter UC emission compared to LaF₃: Yb/Er. Moreover, Capobianco's group had done a pioneering UC study on Yb/Tm co-doped BaYF₅ nanoparticles and confirmed the energy transfer between Yb³⁺ and Tm³⁺ ions mediated by phonon [13]. Compared with the previously reported BaYF₅ and NaYF₄ UCNPs, the Ln³⁺ doped BaGdF₅ UCNPs may not only exhibit excellent UC emission, but also present attractive paramagnetic property owing to the large magnetic moment of Gd³⁺, which makes the Ln³⁺ doped BaGdF₅ as a potential fluorescent and magnetic probe for biomedical application. Recently, Lin's group reported a thermal decomposition method to synthesize Yb/Er co-doped BaGdF₅ NPs with active core/shell structure, showing more efficient UC emission than that of hexagonal phase NaYF₄ [14]. Our previous report also revealed that BaGdF₅ is one type of promising multifunctional UC hosts [15]. Unfortunately, the reported BaGdF₅ is hydrophobic, thereby limiting its use for fluorescent bioimaging application. So far, there is no report on the synthesis of water-soluble BaGdF₅ nanoparticles via a simple and one-pot method. Moreover, no effort was made to employ BaGdF₅ host based NPs with small size on the application in fluorescent bioimaging, especially in dual-modal fluorescent/magnetic bioimaging application.

Apart from fluorescent/magnetic bioimaging, CT is a well-established clinical diagnosis technique that is capable of providing high-resolution 3D information of the anatomic structure of tissues based on the differential X-ray absorption ability of the tissues. However, owing to the low sensitivity to soft tissues, its applications in disease detection have been greatly limited. In contrast to CT, magnetic resonance imaging (MRI) can provide unsurpassed 3D soft tissue details and functional information due to the non-ionizing radiation. Although CT and MRI techniques possess many advantages, both of them suffer from limited planar resolution and are not suitable for cellular level imaging, which can be solved by fluorescent imaging. Therefore, a synergistic combination of fluorescence, CT and MRI contrast agents in single system, though can help combine the advantages of each while avoiding the disadvantages of the other, the making of which faces a great challenge.

So far, there are only a few trimodal nanoprobes for bioimaging. For instance, a fluorescence/CT/MRI trimodal system based on paramagnetic CdS: Mn/ZnS quantum dots (QDs) was reported. [16] However, these QDs suffer from some inherent problems including the high toxicity and low tissue penetration owing to the excitation of ultraviolet (UV) light, which limited their application as imaging probes.

Compared with the conventional fluorescence probes, such as organic dyes and QDs, near-infrared (NIR)-excited upconversion nanoparticles (UCNPs) possess many advantages, including low-autofluorescence, deep tissue penetration, large anti-Stokes shifts, high photostability, and low toxicity. Among all of the developed UC hosts, fluorides are considered as the most efficient host lattice for UC luminescence owing to their low phonon energy. Most reports have been focused on the development of lanthanide doped NaYF₄ UCNPs for fluorescent bioimaging of cells and tissues in vitro and in vivo.

Very recently, a PEGylated NaY/GdF₄: Yb, Er, Tm@SiO₂—Au@PEG₅₀₀₀ system for trimodal bioimaging was designed by using co-thermolysis method in non-hydrolytic solvents and multi-step synthetic procedures [17]. However, these hydrophobic NPs synthesized by the co-thermolysis method also need further surface modification, and the multi-step experiment procedures make the experiment laborious and complex, thereby limiting its use for bioimaging applications. Therefore, it is of very importance to find a new trimodal fluorescence/CT/MRI imaging probes by a simple method in single phase material. To the best of our knowledge, trimodal fluorescence/CT/magnetic nanoprobe based on lanthanide doped BaGdF₅ host materials has not been exploited yet. Two recent reports by Zeng et al. [18,19] have reported two types of modified UCNPs having a host lattice structure of BaGdF₅ co-doped with Yb/Er, and the disclosures of which are incorporated herein by reference.

### SUMMARY OF THE INVENTION

The first aspect of the present invention relates to a water soluble, single-phase and non-hydrophobic bioprobe for multi-modal bioimaging based on surface-modified BaGdF₅:Yb/Er upconversion nanoparticles (UCNPs). The modified UCNPs of the present invention are synthesized by a one-pot hydrothermal method with surface modification by capping different functional groups including but not limited to poly(ethylene glycol) (PEG) moiety, amino group and carboxyl group. The surface modification is performed simultaneously with the synthesis of the UCNPs. In other words, no post-synthesis surface modification is required in the present invention. The size of each nanoparticle of the modified UCNPs in the present invention ranges from 8-15 nm. The modified UCNPs of the present invention can be used as an upconversion fluorescent dye in fluorescence bioimaging because of the upconversion luminescent property (i.e. being excited by near-infrared light at wavelength of about 980 nm); the modified UCNPs can also be used as a contrast agent for MRI because of the paramagnetic property of Gd³⁺ in the host lattice of the UCNPs; the modified UCNPs can also be used as a contrast agent for CT imaging because of different X-ray absorption coefficients of two elements, Ba and Gd, in the host lattice at different photon energy levels as well as the ability to provide a long-lasting enhancement of signal and long circulation time in the recipient of the UCNPs. The modified UCNPs also possess excellent cell penetrating ability such that it facilities internalization of the bioprobe in the target cells or tissues for in vivo bioimaging.

The second aspect of the present invention relates to a method of preparing the modified BaGdF₅:Yb/Er UCNPs. A simple one-pot hydrothermal method is employed in the present invention to prepare the modified UCNPs. A solvent containing at least one surface modifying agent is first provided. In one embodiment, polyethylenimine (PEI) is dissolved in ethylene glycol (EG, 99%) in a concentration of 75 g/L. In another embodiment, poly(ethylene glycol) (PEG) methyl ether is dissolved in ethylene glycol in a concentration of 75 g/L. The choice of surface modifying agent depends on the purpose of the nanoparticles. Other compounds such as 3-mercaptopropionic acid and 6-aminocaproic acid can also be used as the surface modifying agent in the present invention, or a mixture of more than one surface modifying agent. After that, compounds of lanthanide which form the host lattice of the UCNPs are agitated thoroughly at a defined molar ratio in the solvent containing the surface modifying agent to form a first mixture. In one embodiment, the lanthanide compounds includes the formula of Ln(NO₃)₃.6H₂O or Ln(Cl₃)₃.6H₂O, where Ln is Gd, Yb, or Er. In other embodiment, the lanthanide compounds include Gd(NO₃)₃, Yb(NO₃)₃, and Er(NO₃)₃ and the molar ratio of these compounds is 78:20:2 or 80:18:2. BaCl₂ is added to the first mixture and further agitated for 30 minutes until a homogeneous solution is formed. Ethylene glycol containing NH₄F is then added to the homogeneous solution and agitated for another 30 minutes to form a reaction mixture. The reaction mixture is then kept in an autoclave at 190° C. for 24 hours. After cooling down naturally to room temperature from autoclave, the particles formed in the reaction mixture are separated by centrifugation and then washed several times with ethanol and water to remove residual solvents before drying in a vacuum. The resulting nanoparticles after drying are ready for use which does not require additional surface modification because their surface has been modified during the series of mixing and reaction of different compounds.

The third aspect of the present invention relates to methods of using the modified UCNPs of the present invention for multi-modal bioimaging including fluorescent imaging, magnetic application (e.g. magnetic resonance imaging or MRI) and computed X-ray tomography (CT) imaging. In one embodiment, the modified UCNPs of the present invention are used as an upconversion fluorescent probe in vitro or in vivo. Because of the upconversion property, the modified UCNPs can be excited using near-infrared (NIR) at about 980 nm instead of using high-energy light source which is commonly used in the conventional fluorescent probe. A green fluorescent signal is generated by the modified UCNPs under the excitation of NIR while a relatively weaker red fluorescent signal is also generated simultaneously when it is used to imaging cells. Lanthanide (Ln³⁺) co-doped BaGdF₅ nanoparticles do not only exhibit excellent upconversion property but also possess paramagnetic property owing to the large magnetic moment of Gd³⁺, which makes the Ln³⁺ doped BaGdF₅ as a potential magnetic probe for biomedical application. Both barium (Ba) and gadolinium (Gd) are promising CT contrast elements owing to their large K-edge values and high X-ray mass absorption coefficients. Therefore, the BaGdF₅ host containing binary CT contrast elements (Ba, Gd) having different X-ray mass absorption coefficients becomes a potential CT imaging contrast agent at various photon energy to suit different clinical applications. The modified UCNPs of the present invention also provides a long-lasting enhancement of signal and long circulation time in vivo when it is used as a CT contrast agent. The optimal concentration of the modified UCNPs used for bioimaging in cells or tissues is in a concentration of about 100 to 1,000 μg/mL. For in vivo bioimaging, the modified UCNPs of the present invention are administered to a subject in needs thereof through different routes including but not limited to subcutaneous, intravenous, and intramuscular routes. Other possible administration routes may be used for delivering said modified UCNPs to the subject if appropriate.

### BRIEF DESCRIPTION OF THE DRAWINGS

FIG. 1 is FTIR spectra of (a) the amine-functionalized BaGdF₅:Yb/Er UCNPs and (b) the PEG-modified BaGdF₅:Yb/Er UCNPs.

FIG. 2 are TEM and XRD results of the PEG-modified BaGdF₅:Yb/Er UCNPs: (a) Typical TEM image, (b) Corresponding SAED pattern, (c) HRTEM image, (d) XRD pattern, (e) EDS. (inset of FIG. 2a indicates the histogram of the particle size distribution).

FIG. 3: (a) Upconversion spectra of the PEG-modified BaGdF₅:Yb/Er NPs; (b) The Log-Log plots of the UC luminescence intensity versus excitation power, the inset of FIG. 3a shows photograph of the water colloidal solutions of UCNPs (1 wt %) excited by 980 nm laser diode; (c) Simplified energy-level diagrams of Yb³⁺/Er³⁺.

FIG. 4: In vitro bioimaging of the amine-functionalized BaGdF₅:Yb/Er colloidal UCNPs in HeLa cells: (a) bright field image of HeLa cells, (b) corresponding green UC fluorescent image (500-600 nm), (c) the red emission UC fluorescent image (600-700 nm). The concentration of UCNPs was 100 μg/mL and the incubation time was 24 hours.

FIG. 5: In vitro fluorescence imaging of HeLa cells excited by a 980 nm laser with different excitation power after incubated with the amine-functionalized BaGdF₅:Yb/Er colloidal UCNPs: (a) bright field image, and: (b) 300 mW, (c) 500 mW, (d) 800 mW, (e) the corresponding visible up-converted in-vitro emission spectra obtained from FIG. 5d. The concentration of UCNPs was 100 μg/mL and the incubation time was 24 hours.

FIG. 6: In vitro bioimaging of the PEG-modified BaGdF₅:Yb/Er colloidal UCNPs in HeLa cells: (a) bright field image of HeLa cells, (b) corresponding green UC fluorescent image (500-600 nm), (c) the red emission UC fluorescent image (600-700 nm). The concentration of UCNPs was 150 μg/mL and the incubation time was 24 hours.

FIG. 7: MTT assay for cytotoxicity of the amine-functionalized BaGdF₅:Yb/Er UCNPs in HeLa cells. The amine-functionalized BaGdF₅:Yb/Er UCNPs were incubated with HeLa cells at 37° C. for 24 hours.

FIG. 8: In vitro cell viability of HeLa cells incubated with different concentrations of the PEG-modified BaGdF₅:Yb/Er UCNPs at 37° C. for 24 hours under 5% CO₂.

FIG. 9: (a) Relaxation rate R1 (1/T1) versus various molar concentrations of hydrophilic BaGdF₅:Yb/Er NPs at room temperature using a 3 T MRI scanner, (b) T₁-weighted images of BaGdF₅:Yb/Er NPs with different concentrations (mM) in water.

FIG. 10: Magnetization as a function of applied field for the PEG-modified BaGdF₅:Yb/Er UCNPs at room temperature.

FIG. 11: (a) CT images of water solutions under different concentrations of PEG-modified BaGdF₅:Yb/Er UCNPs, (b) the measured CT values (Hounsfield units, HU) of PEG-modified BaGdF₅:Yb/Er UCNPs.

FIG. 12: In vivo X-ray CT imaging of a mouse before and after intravenous injection of 500 μL of PEG-modified BaGdF₅:Yb/Er UCNPs (0.05 M) at different time periods: (a) pre-injection, (b) 5 min, (c) 30 min, (d) 60 min, (e) 120 min. The left panel: maximum intensity projection (MIP), the middle panel: the corresponding 3D volume-rendered (VR) in vivo CT images of mice; the right panel: lateral view of 3D VR CT images.

FIG. 13 X-ray K-edge absorption coefficients of Ba, Gd, and I at different photon energy levels.

### DEFINITIONS

“Upconversion”, or in short “UC”, used herein refers to a process in which the sequential absorption of two or more photons leads to the emission of light at shorter wavelength than the excitation wavelength.

“Nanoparticle” used herein refers to a particle which has an average size of 100 nm to 1 nm, or otherwise specified in the present application.

“Amine-modified” and “Amine-functionalized” used interchangeably herein refers to positively charged amino group being coated on the surface of BaGdF₅:Yb/Er UCNPs of the present invention.

### DETAILED DESCRIPTION OF THE INVENTION

In the present invention, simultaneous synthesis and surface functionalization of BaGdF₅:Yb/Er UCNPs by a simple, facile and one-pot hydrothermal method is employed to synthesize the modified UCNPs of the present invention. Water and some low toxic organic agents are used as reaction media in the present invention, which have not been used in any of the conventional method. The synthesized UCNPs of the present invention have small size range of 8-15 nm which are well dispersed in polar solutions, such as water and ethanol.

In one embodiment, the amine-functionalized BaGdF₅:Yb/Er UCNPs have an average particle size of about 10 nm. In another embodiment, the PEG-modified BaGdF₅:Yb/Er UCNPs have an average particle size of about 12 nm which is slightly larger than the amine-functionalized UCNPs because of the presence of the PEG moiety on the surface of the nanoparticle. Both embodiments of the modified UCNPs are an ideal bioprobe for fluorescent imaging, T₁-weighted MRI application and computed X-ray tomography. Owing to positively charged amino group (+27.6 mV) on the surface, the amine-functionalized UCNPs have high water solubility and are feasible to enter into the cells. The amine-functionalized UCNPs are also an effective fluorescent label in imaging cells because the local fluorescence ascribed to the energy transition of Er³⁺ ion has been observed from fluorescent microscopy. The amine-functionalized UCNPs also possess low toxicity. Moreover, the amine-functionalized UCNPs present an excellent paramagnetic property and relatively large longitudinal relaxivity of 1.194 S⁻¹ mM⁻¹. More importantly, the amine-functionalized UCNPs can also be used as T₁ MRI contrast agent. Consequently, the amine-functionalized BaGdF₅: Yb/Er UCNPs with low toxicity are a promising multi-modal bioprobe.

The PEG-modified UCNPs, like amine-functionalized UCNPs, are also an ideal bioprobe for tri-modal bioimaging. The PEG-modified UCNPs can be used as fluorescent bioprobes under the excitation of near infrared (NIR) laser and have low cytotoxicity to HeLa cells. In addition, the PEG-modified UCNPs also present an excellent paramagnetic property which can be used for various biomagnetic applications, e.g. as a contrast agent for MRI. The PEG-modified UCNPs are also a powerful CT contrast agent because the signals of which in water solution are significant due to the presence of two contrast elements (Ba and Gd) in the host lattice of the modified UCNPs which have different absorption coefficients at different photon energies (at 60 keV, Ba: 8.51 cm² g⁻¹, Gd: 1.18 cm² g⁻¹; at 80 keV, Ba: 3.96 cm² g⁻¹, Gd: 5.57 cm² g⁻¹) and large K-edge values (Ba_{K-edge}: 37.4 keV, Gd_{K-edge}: 50.2 keV). Moreover, the PEG-modified UCNPs possess long-lasting enhancement of signal in vivo, e.g. to keep a significant signal level for about 2 hours in vivo. More importantly, the long circulation time in vivo of the PEG-modified UCNPs, e.g. for about 2 hours in blood circulation when it is administered via subcutaneous, intravenous, or intramuscular route, can help the detection of various diseases (e.g. splenic diseases) and imaging of targeted tumor. Owing to different X-ray absorption coefficients of Ba and Gd, the PEG-modified BaGdF₅:Yb/Er UCNPs as a CT contrast agent can be used at different operating voltages for various clinical application purposes.

Also disclosed in the present invention are methods of using the modified UCNPs of the present invention for tri-modal bioimaging. The modified UCNPs of the present invention can be used as an upconversion fluorescent dye, MRI contrast agent, and CT contrast agent.

In the following examples, the in vitro fluorescent bioimaging of HeLa cells is demonstrated by using near-infrared (NIR) to visual UC transition of the modified BaGdF₅:Yb/Er UCNPs. The measurement of cytotoxicity assay demonstrates that the modified BaGdF₅:Yb/Er UCNPs have low toxicity in HeLa cells. More importantly, owing to the paramagnetic property of Gd³⁺ in the host lattice of BaGdF₅, the T₁-weighted magnetic resonance imaging (MRI) is also achieved, making the modified BaGdF₅:Yb/Er UCNPs as a promising MRI contrast agent. Most importantly, the in vitro and in vivo CT imaging result shows the excellent ability in visualizing tissue of animal, e.g. the spleen tissue of small animal, by the modified UCNPs owing to different absorption coefficients of Ba and Gd at different photon energy levels, which suggests that the modified BaGdF₅: Yb/Er UCNPs can also be used as a CT contrast agent.

### EXAMPLES

The present invention is now explained more specifically by referring to the following examples. These examples are given only for a better understanding of the present invention, and not intended to limit the scope of the invention in any way.

### Example 1

### Chemicals and Materials

Ln(NO₃)₃.6H₂O or Ln(Cl₃)₃.6H₂O (Ln=Gd, Yb, Er,) was purchased from Aldrich and dissolved in de-ionized water (DI-water) to form solution with concentration of 0.5 M and 0.1 M. Ethylene glycol (EG, 99%) and branched polyethylenimine (PEI, 25 kDa) were purchased from Sigma-Aldrich; Poly(ethylene glycol) methyl ether (PEG, average molecular=5000) was purchased from Sigma-Aldrich. NH₄F (99.99%) and BaCl₂ (99.99%) were obtained from Sinopharm Chemical Reagent Co., China. All of these chemicals were used as received without further purification.

### Example 2

### One-Pot Synthesis of Amine-Functionalized or PEG-Modified BaGdF₅:Yb/Er UCNPs

The water-soluble, single-phase and non-hydrophobic modified BaGdF₅:Yb/Er UCNPs with high monodispersity were synthesized by a modified one-pot hydrothermal method. In this example, 1.5 g of PEI or 1.5 g of PEG methyl ether were added into 20 mL EG containing 1 mmol of Gd(NO₃)₃ (0.5 M), Yb(NO₃)₃ (0.5 M) and Er(NO₃)₃ (0.1 M) with the molar ratio of 78:20:2 (for amine-modified UCNPs) or 80:18:2 (for PEG-modified UCNPs) under vigorous stirring to form a first solution. Then, 1 mmol of BaCl₂ was added to the first solution and stirred for 30 min to form a homogeneous solution. After that, 5.5 mmol of NH₄F dissolved in 10 mL of EG was added to the homogeneous solution and agitated for another 30 min, and then transferred into a 50 mL stainless Teflon-lined autoclave and kept at 190° C. for 24 hours. After the 24-hour reaction, the reaction mixture was naturally cooled down to room temperature. The prepared samples (particles) were separated by centrifugation, washed for several times with ethanol and DI-water to remove other residual solvents, and finally dried in vacuum at 60° C. for another 24 hours. The dried particles (i.e. the amine-modified UCNPs) were obtained for further characterization.

### Example 3

### Characterization of the Modified BaGdF₅:Yb/Er UCNPs

To study the phase composition of the modified UCNPs, powder X-ray diffraction (XRD) patterns of the modified UCNPs obtained from Example 2 were recorded using a Bruker D8 advance X-ray diffractometer at 40 KV and 40 mA with Cu—Kα radiation (λ=1.5406 Å). The shape, size and structure of the modified UCNPs were characterized by using JEOL-2100F transmission electron microscopy (TEM) equipped with an Oxford Instrument EDS system, operating at 200 kV. To study the surface structure of the modified UCNPs, Fourier transform infrared spectrum (FTIR) was recorded by a Magna 760 spectrometer E. S. P. (Nicolet). ξ-potential measurement was performed on a Zetasizer 3000 HAS (Malven Instruments, UK). Photoluminescence/UC spectra of the modified UCNPs were recorded using FLS920P Edinburgh analytical instrument apparatus equipped with 980 nm diode laser as an excitation source. The magnetization of the modified UCNPs was measured as a function of the applied magnetic field ranging from −20 to 20 kOe at room temperature (RT) using a Lakeshore 7410 vibrating sample magnetometer (VSM).

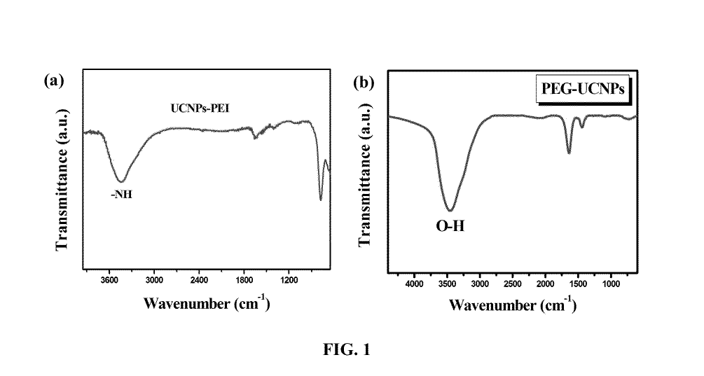

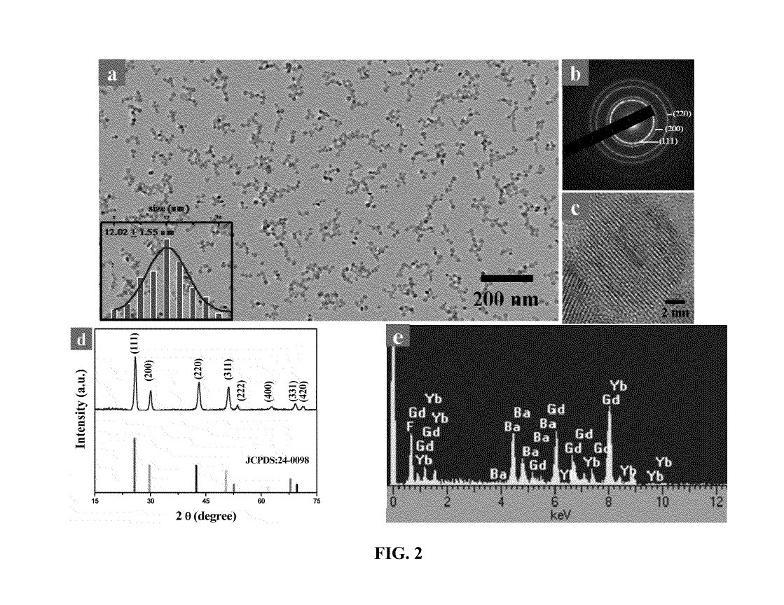

Earlier studies indicated that the positively charged amino group coated on the surface of the amine-functionalized UCNPs does not only increase their water-solubility but also greatly enhance cellular uptake. In contrast, some neutral and negative polymers, such as polyvinylpyrrolidone (PVP) and poly(acrylic acid) (PAA), do not possess the properties necessary for multi-modal bioimaging. By considering the fact, polyethylenimine (PEI) is used as a surface modifying agent for amine functionalization of BaGdF₅:Yb/Er UCNPs. Water soluble and amine-functionalized BaGdF₅:Yb/Er UCNPs are synthesized via a simple and facile one-pot hydrothermal method by using PEI as a capping ligand. The c-potential for the UCNPs colloidal solution is around +27.6 mV, indicating the successful conjugation of positively charged PEI on the surface of nanoparticles. Moreover, the presence of the amino group on the surface of amine-functionalized UCNPs is further verified by FTIR spectrum (FIG. 1a). A broad band at about 3,449 cm⁻¹ related to the amine groups (NH) stretching vibration further indicates that PEI molecules have successfully been coated on the surface of nanoparticles. The FTIR spectra for the PEG-modified UCNPs (FIG. 1b) show a broad band centered at 3,451 cm⁻¹ attributed to the O—H stretching vibration, indicating that the PEG molecules have successfully been grafted on the surface of nanoparticles. Transmission electron microscopy (TEM) image (FIG. 2a) demonstrates that the PEG-modified UCNPs have sphere shape and high monodispersity. The PEG-modified UNCPs possess average size of 12.02±1.55 nm according to the size-distribution obtained from TEM images (inset of FIG. 2a). FIG. 2b shows the corresponding selected area electron diffraction (SAED) pattern, indicating that the PEG-modified UCNPs are face-centered cubic (FCC) phase structure. To further reveal the structure of PEG-modified UCNPs, the high-resolution TEM (HRTEM) image of a single NP was investigated. As shown in FIG. 2c, a clearly lattice fringe with a measured d-spacing of about 2.1 Å was observed, matching the (220) lattice plane of cubic phase BaGdF₅. The powder X-ray diffraction (XRD) was used to reveal the phase composition of the PEG-modified UCNPs. As shown in FIG. 2d, the diffraction peaks can be readily indexed to FCC phase structure (JCPDS 24-0098) and no other impurity peaks were observed, indicating the formation of pure cubic phase BaGdF₅ and a homogeneous Gd—Yb solid solution structure. Moreover, owing to the substitution of Gd³⁺ by smaller Yb³⁺, the diffraction peaks shift to higher angle direction in XRD pattern. As shown in FIG. 2e, the energy dispersive X-ray spectroscopy (EDS) of the as-prepared UCNPs demonstrates that the compositions of UCNPs are Ba, Gd, F, and the dopant Yb, providing further evidence on the incorporation of Yb³⁺ into BaGdF₅ host matrix. Notably, the signals of C and Cu are attributed to the TEM copper grid and the covered carbon film on the supporting copper, respectively.

### Example 4

### Upconversion Properties of the Modified UCNPs

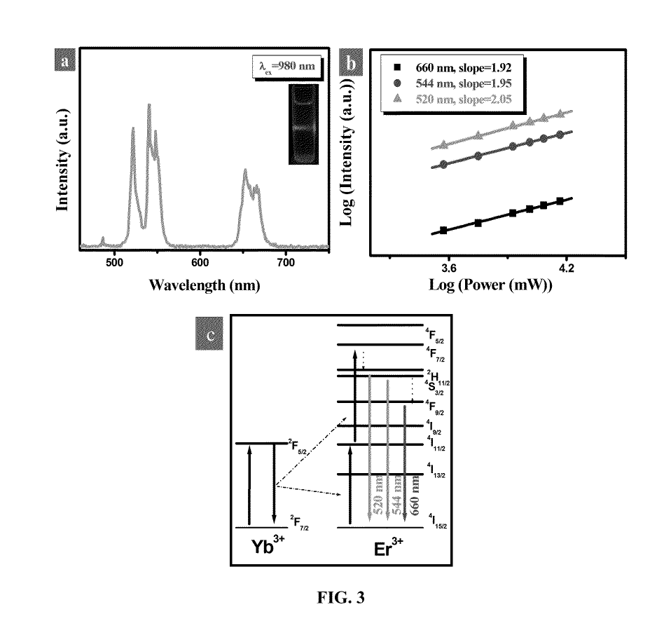

The UC property of the PEG-modified UCNPs was also demonstrated by the UC emission spectra recorded under the excitation of a 980 nm laser diode (LD) at room temperature (RT). The photography image (the inset of FIG. 3a, 1 wt % water colloidal solutions of UCNPs) demonstrated that the PEG-modified UCNPs emit bright and eye-visible green UC emission. FIG. 3a shows the typical UC luminescence spectra of the PEG-modified BaGdF₅:Yb/Er UCNPs. The intense green and red emission bands centered at 521, 544, and 660 nm were observed, respectively. According to the simplified energy level diagram (FIG. 3c), the green emission band of Er³⁺ ion centered at 521/544 nm was attributed to the electronic transition ²H_{11/2}/⁴S_{3/2}→⁴I_{15/2} ion while the 660 nm red emission was attributed to the ⁴F_{9/2}→⁴I_{15/2} energy transition. To further reveal the UC mechanism, the excitation power dependent UC emissions of green and red bands were investigated. Generally, the output UC luminescent intensity (I_{UC}) is proportional to the infrared excitation (I_{IR}) power via the following formula:

I_{UC}∝I_{IR}ⁿ,

where n is the number of absorbed photon numbers for per visible photon emitted and its value can be obtained from the slope of the fitted line in the plot of log I_{UC} versus log I_{IR}. As shown in FIG. 3b, the slopes of the linear fit for the green and red emissions at 520, 544 and 660 nm are 2.05, 1.95 and 1.92, respectively, implying that a two-photon process is involved in both green and red UC luminescence.

### Example 5

### Cell Culture

Human cervical carcinoma HeLa cells were purchased from the American type Culture Collection (ATCC) (#CCL-185, ATCC, Manassas, Va., USA). The HeLa cells were grown in Dulbecco's Modified Eagle Medium (DMEM) supplemented with 10% fetal bovine serum (FBS) 1% penicillin and streptomycin at 37° C. and 5% CO₂. To apply the amine-modified or PEG-modified UCNPs for fluorescent imaging, HeLa cells were incubated in DMEM containing 100-5,000 μg/mL of the amine-modified or PEG-modified UCNPs at 37° C. for 24 hours under 5% CO₂, and then washed with PBS sufficiently to remove excess nanoparticles.

### Example 6

### In Vitro Bioimaging

To test the suitability of the obtained amine-modified UCNPs as bioprobes, bioimaging of HeLa cells incubated with the amine-modified UCNPs was performed on a commercial con-focal laser scanning microscope-Leica TCS SP5 equipped with a Ti: Sapphire laser (Libra II, Coherent). The samples containing HeLa cells and the amine-modified UCNPs were excited by a 980 nm wavelength laser, and two visible upconversion emission channels were detected at green (500-600 nm) and red (600-700 nm) spectral regions.

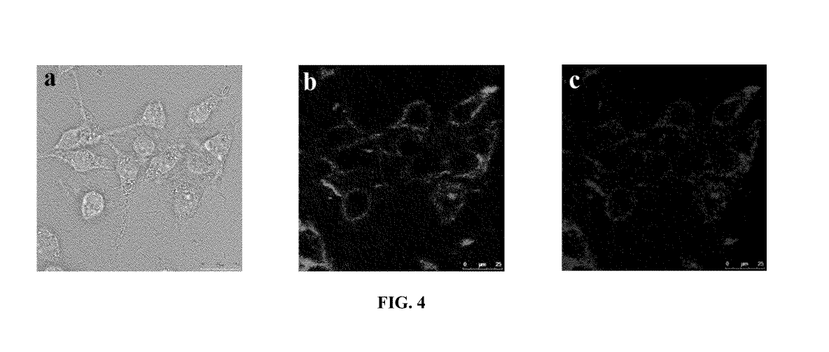

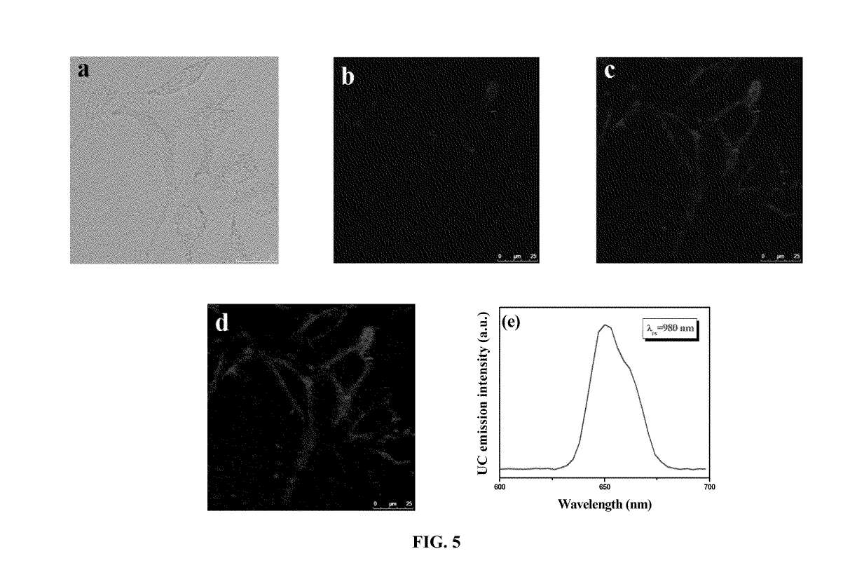

It is clearly shown in FIG. 4b that the cells incubated with the amine-functionalized UCNPs exhibited bright green UC fluorescence, confirming the cell uptake of the amine-functionalized UCNPs. A relatively weaker red UC fluorescence is also observed in the cell membrane, as shown in FIG. 4c. These results indicate that the amine-modified UCNPs can be encapsulated into human cervical carcinoma cells, and the UC fluorescence is strong enough for the cell imaging. Compared with the green UC emission, the red UC emission is relatively weak. FIG. 5 shows the effect of incident laser power on the bioimaging of HeLa cells. As increasing the excitation power of the 980 nm laser, the red UC signal was also gradually increased, which is in good agreement with previous reports. FIG. 5e are the UC emission spectra excited under 980 nm laser obtained from the area in FIG. 5d. This result further supports that the modified BaGdF₅:Yb/Er UCNPs of the present invention have successfully incorporated into HeLa cells. Moreover, owing to the unique UC character of UCNPs, no autofluorescence could be detected when increasing the laser power up to 800 mW (FIGS. 5d and 5e), resulting in a high signal-to-noise ratio.

PEG-modified UCNPs with concentration of 150 μg/mL were incubated with HeLa Cells at 37° C. for 24 hours under 5% CO₂. After washed with PBS for three times, upconversion fluorescent imaging of HeLa Cells was performed in vitro on a commercial con-focal laser scanning microscope-Leica TCS SP5 equipped with a Ti: Sapphire laser (Libra II, Coherent). The samples containing cells with PEG-modified UCNPs were excited by a laser of 980 nm wavelength, and two visible UC emission signals were detected at green (500-600 nm) and red (600-700 nm) regions.

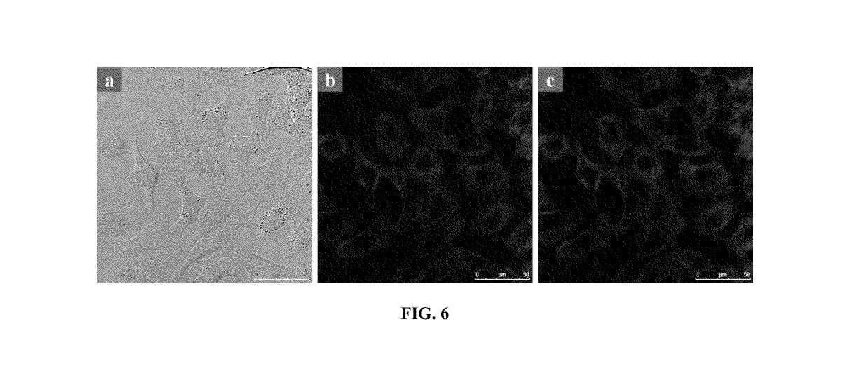

As shown in FIG. 6, the cells exhibited bright green and red UC fluorescence, indicating the internalization of the PEG-modified UCNPs in HeLa cells.

### Example 7

### Cytotoxicity Assay

The in vitro cell viability was measured using 3-(4,5-dimethylthiazol-2-yl)-2,5 diphenyl-tetrazolium bromide (MTT) proliferation assay on HeLa cells pre-incubated with different concentrations of amine-modified UCNPs from 100 to 5,000 μg/mL. HeLa Cells were seeded into a 96-well micro-plate (6000 cells/well) and pre-incubated at 37° C. under 5% CO₂ for 3 hours. The cell culture medium in each well was replaced by DMEM solutions containing different concentrations of amine-modified UCNPs. Subsequently, the cells were incubated for another 20-24 hours in the incubator at 37° C. under 5% CO₂. And then 10 μL MTT (5 mg/mL in phosphate buffered saline solution) was added to each well and further incubated for 4 hours at 37° C. under 5% CO₂. After removing the PBS, 200 μL of DMSO was added to each well, sitting at room temperature overnight to dissolve the formazan crystals completely. The absorbance at 570 nm was measured by Multiskan EX (Thermo Electron Corporation).

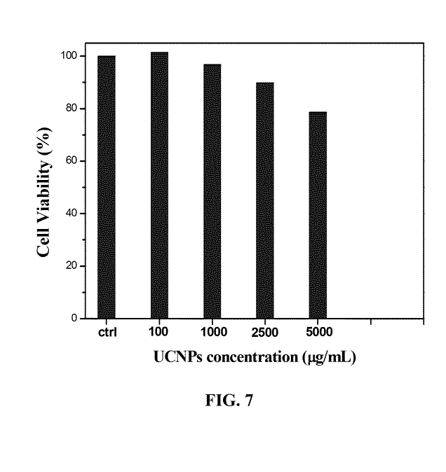

In FIG. 7, cell viability is greater than 90% when 100 μg/mL of amine-modified BaGdF₅:Yb/Er UCNPs is used in cell imaging. When further increasing the concentration of the amine-modified BaGdF₅:Yb/Er UCNPs up to 1,000 μg/mL, cell viability is still greater than 95%, indicating the cytotoxicity of BaGdF₅:Yb/Er UCNPs is low. All of these results demonstrate that the amine-functionalized BaGdF₅: Yb/Er UCNPs are promising as fluorescent probes for bioimaging with the features of autofluorescence free and low cytotoxicity.

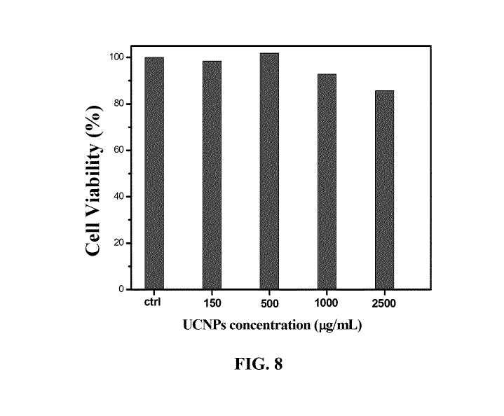

The cell viability of HeLa Cells incubated with PEG-modified UCNPs in different concentrations of 150, 500, 1,000, and 2,500 μg/mL was also measured by MTT assay. FIG. 8 shows that cell viability is greater than 86% when the concentration of our PEG-modified BaGdF₅: Yb/Er UCNPs is increased up to 2,500 μg/mL, indicating the cytotoxicity of PEG-modified BaGdF₅: Yb/Er UCNPs is very low. As a result, these multi-functional PEG-modified BaGdF₅: Yb/Er UCNPs are promising as UC fluorescent probes for bioimaging with low cytotoxicity.

### Example 8

### Measuring Relaxation Properties of BaGdF₅:Yb/Er UCNPs as MRI Contrast Agent

Apart from the excellent UC property, owing to the large magnetic moment of Gd³⁺ included in the new host of BaGdF₅, the amine-functionalized BaGdF₅:Yb/Er UCNPs could act as a T₁ MRI contrast agent as well. The relaxation property of the amine-functionalized BaGdF₅:Yb/Er UCNPs was characterized on a 3T Siemens Magnetom Trio by detecting the longitudinal relaxation times (T₁) using a standard inversion-recovery (IR) spin-echo sequence. The molar relaxivity 1/T₁ (R1) can be determined by the slope of the following equation.

(1/T₁)_{obs}=(1/T₁)_{d}+R1[M]

where (1/T₁)_{obs} and (1/T₁)_{d} are the observed values in the presence and absence of BaGdF₅ UCNPs, respectively. [M] is the concentration of BaGdF₅ UCNPs.

The T₁-weighted MRI images were acquired at room temperature using a 3T Siemens Magnetom Trio. Various concentrations of amine-functionalized BaGdF₅:Yb/Er UCNPs (0, 0.2, 0.4, 0.8 mM) water solutions were put in a series of 1.5 mL tubes for T₁-weighted MRI with a T₁-weighted sequence.

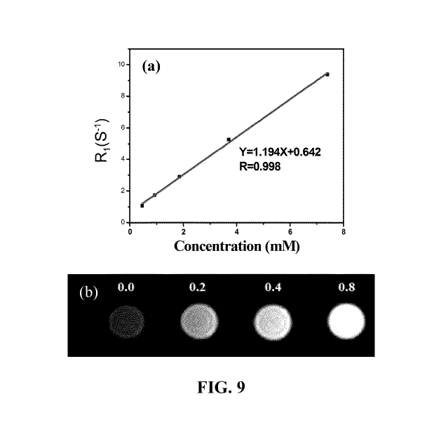

According to our previous study [18], the paramagnetic properties of the Gd³⁺ ions in the amine-functionalized UCNPs come from seven unpaired inner 4f electrons, which are closely bound to the nucleus and effectively shielded by the outer closed shell electrons 5s²5p⁶ from the crystal field. The magnetic mass susceptibility of the amine-functionalized UCNPs is found to be 4.72×10⁻⁵ emu/gOe. The magnetization of UCNPs is around 0.95 emu/g at 20 kOe, which is close to the value reported for nanoparticles used for common bioseparation. To further demonstrate the amine-functionalized UCNPs as potential MRI contrast agent, a series of amine-functionalized UCNPs with different molar concentrations were used for the ionic longitudinal relaxivity (R1) study under a 3 T MRI scanner. From the slope of the concentration-dependent relaxation rate 1/T₁ (R1) (FIG. 9a), R1 value for the amine-functionalized UCNPs was determined to be 1.194 S⁻¹·mM⁻¹. FIG. 9b shows typical T₁-weighted MRI. When increasing the concentration of amine-functionalized UCNPs, the T₁-weighted MRI signal intensity was clearly enhanced, demonstrating that Gd³⁺-containing UCNPs is an effective T₁ MRI contrast agent. Therefore, this result has provided a simple strategy for combining two functions incorporating fluorescent and magnetic properties into a single compound (BaGdF₅:Yb/Er), eliminating the need for complicated procedures.

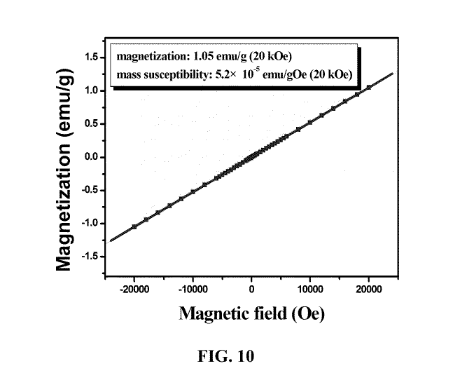

Similarly, the excellent paramagnetic nature of the PEG-modified UCNPs is shown in FIG. 10, which is mainly attributed to the seven unpaired inner 4f electrons of Gd³⁺. The magnetization and mass susceptibility of the PEG-modified BaGdF₅ UCNPs are around 1.05 emu/g and 5.2×10⁻⁵ emu/gOe at 20 kOe, respectively, which is close to the value reported for nanoparticles used for MRI contrast agent, and common bioseparation.

### Example 9

### In Vitro and In Vivo CT Imaging

Due to the high X-ray absorption coefficient of Ba and Gd, the PEG-modified BaGdF₅:Yb/Er UCNPs should have the potential in the use of promising nanoparticle-based CT contrast agents. To validate CT contrast efficacy, X-ray CT phantom images were acquired using different concentrations of PEG-modified BaGdF₅: Yb/Er in deionized water at 60 keV. Different concentrations of PEG-modified BaGdF₅:Yb/Er UCNPs (0, 5, 10, 20, 40, 80 mM) were dispersed in de-ionized water for in vitro CT imaging. In order to study the in vivo CT imaging, a mouse was first anesthetized by intraperitoneal injection of chloral hydrate solution (10 wt %), and then 500 μL, physiological saline solutions containing the PEG-modified BaGdF₅: Yb/Er UCNPs (0.05 M) were intravenously injected into the mouse via the mouse's caudal vein. CT images were acquired using ZKKS-MCT-Sharp (Chinese Academy of Sciences and Guangzhou Kaisheng Medical Technology Co., Ltd.) as following parameters: thickness, 0.14 mm; pitch, 0.07; 60 KVp, 0.5 mA; large field view; gantry rotation time, 0.5 s; speed, 5 mm/s.

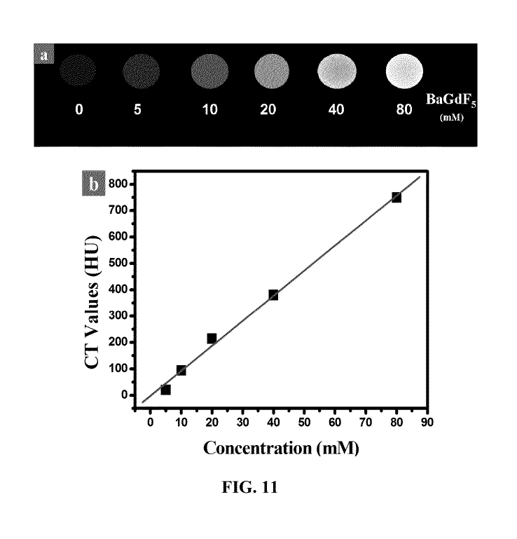

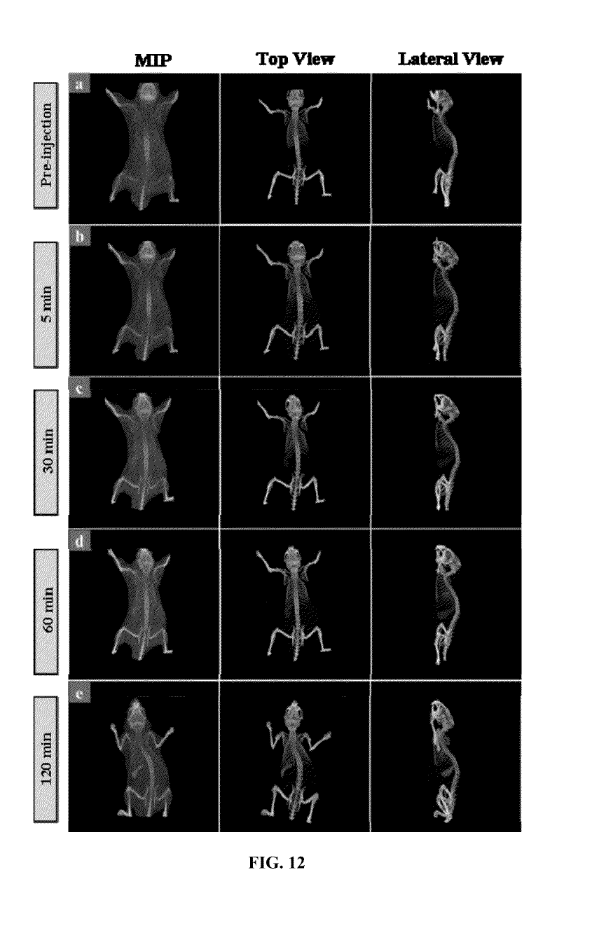

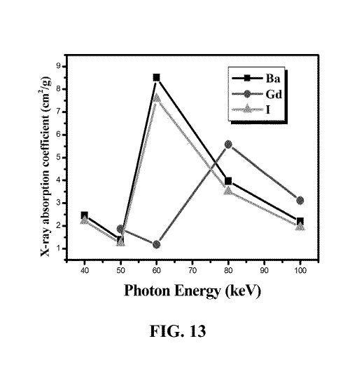

As shown in FIG. 11a, when increasing the concentrations of the agent, the signal was gradually enhanced. In this connection, the measured CT numbers (FIG. 11b), called Hounsfield units (HU), increased linearly with increasing the concentration of the PEG-modified BaGdF₅: Yb/Er UCNPs, indicating the feasibility of the PEG-modified BaGdF₅: Yb/Er as CT contrast agent. To further reveal the feasibility of PEG-modified BaGdF₅: Yb/Er as CT imaging probes, a mouse intravenously administered a amount of PEG-modified BaGdF₅: Yb/Er UCNPs solution (500 μL, 0.05 M) was detected by X-ray CT imaging at different injecting time (FIG. 12). As shown in the pre-injection image (FIG. 12a), no soft tissues can be rendered by X-ray CT imaging. After intravenously injected for 5 min, a weak signal of spleen can be observed from the 3D volume-rendered (VR) CT image (FIG. 12b). With further increasing the time from 30 min (FIG. 12c) to 60 min (FIG. 12d), a significant enhancement of the signal of the spleen could be observed. After 120 min (FIG. 12e), the spleen signal is still obviously observed, indicating these UCNPs can be used as potential imaging probes for the detection of splenic diseases. It should be emphasized that the long-lasting enhancement of the signal may improve the detection of diseases. Interestingly, owing to the different absorption coefficients of Ba, Gd at different photon energies in our developed host (FIG. 13) [20], these PEG-modified BaGdF₅:Yb/Er UCNPs combined two contrast elements (Ba, Gd) meet the requirements from various groups of patients for diagnostic imaging. In FIG. 13, the maximum x-ray absorption coefficient of Ba element is at 60 keV while the maximum x-ray absorption coefficient of Gd element is at 80 keV. It shows that the modified BaGdF₅ UCNPs as CT contrast agent can achieve high CT contrast efficacy at different photon energy for various diagnostic imaging of various patient groups.

If desired, the different functions discussed herein may be performed in a different order and/or concurrently with each other. Furthermore, if desired, one or more of the above-described functions may be optional or may be combined.

Although various aspects of the invention are set out in the independent claims, other aspects of the invention comprise other combinations of features from the described embodiments and/or the dependent claims with the features of the independent claims, and not solely the combinations explicitly set out in the claims.

It is also noted herein that while the above describes exemplary embodiments of the invention, these descriptions should not be viewed in a limiting sense. Rather, there are several variations and modifications which may be made without departing from the scope of the present invention as defined in the appended claims.

### REFERENCE LIST

[1] Chen Z G, Chen, H L, Hu H, Yu M X, Li F Y, Zhang Q, et al. J Am Chem Soc 2008; 130:3023-3029.
[2] Kong W H, Lee W J, Cui Z Y, Bae K H, Park T G, Kim J H, et al. Biomaterials 2007; 28:5555-5561.
[3] Kumar R, Nyk M, Ohulchanskyy T Y, Flask C A, Prasad P N. Adv Funct Mater 2009; 19:853-859.
[4] Boyer J C, Vetrone F, Cuccia L A, Capobianco J A. J Am Chem Soc 2006; 128:7444-7445.
[5] Wang F, Liu X G. Chem Soc Rev 2009; 38:976-989.
[6] Wang F, Han Y, Lim C S, Lu Y H, Wang J, Xu J, et al. Nature 2010; 463:1061-1065.
[7] Zhou J, Sun Y, Du X X, Xiong L Q, Hu H, Li F Y. Biomaterials 2010; 31:3287-3295.
[8] Wang Z L, Hao J H, Chan H L W, Wong W T, Wong K L. Small 2012; 8:1863-1868.
[9] Li Z Q, Zhang Y, Jiang S. Adv Mater 2008; 20:4765-4769.
[10] Xiong L Q, Chen Z G, Yu M X, Li F Y, Liu C, Huang C H. Biomaterials 2009; 30:5592-5600.
[11] Wang Z L, Hao J H, Chan H L W, Law G L, Wong W T, Wong K L, et al. Nanoscale 2011; 3:2175-2181.
[12] Liu Q, Sun Y, Yang T S, Feng W, Li C G, and Li F Y. J Am Chem Soc 2011, 133: 17122-17125.
[13] Vetrone F, Mahalingam V, Capobianco A. Chem Mater 2009, 21:1847-1851.
[14] Yang D M, Li C X, Li G G, Shang, M M Kang X J, Lin J. J Mater Chem 2011, 21: 5923-5927.
[15] Xu C F, Ma M, Yang, L W, Zeng S J, and Yang Q B. J Lumin 2011, 131: 2544-2549.
[16] Santra S, Yang H, Holloway P H, Stanley J T, Mericle R A S. J Am Chem Soc 2005; 127:1656-1657.
[17] Xing H Y, Bu W B, Zhang S J, Zheng X P, Li M, Chen F, et al. Biomaterials 2012; 33:1079-1089.
[18] Zeng S J, Tsang M K, Chan C F, Wong K L, Fei B, Hao J H. Nanoscale 2012, 4: 5118-5124.
[19] Zeng S J, Tsang M K, Chan C F, Wong K L, Hao J H. Biomaterials 2012, 33:9232-9238.
[20] http://physics.nist.gov/PhysRefData/XrayMassCoef/.

1. A water soluble, single-phase and non-hydrophobic bioprobe for multi-modal bioimaging of fluorescence, magnetic resonance imaging (MRI) and computed X-ray tomography (CT) based on a plurality of nanoparticles with upconversion luminescent property, said nanoparticles comprising barium (Ba), gadolinium (Gd), fluorine (F), ytterbium (Yb), and erbium (Er),
wherein the surface of said nanoparticles is modified by polyethylenimine during a one-pot synthesis of said nanoparticles; and
wherein the nanoparticles are hydrophilic.

2. The bioprobe of claim 1, wherein each of said nanoparticles has an average size of about 8 to 15 nm.

3. The bioprobe of claim 1, wherein each of said nanoparticles comprises a host lattice formed by Ba, Gd and F with a chemical formula of BaGdF₅ which is co-doped with Yb and Er.

4. The bioprobe of claim 3, wherein said host matrix has a face-centered cubic (FCC) phase structure and an inter-plane distance (d-spacing) of about 2.1 Å.

5. The bioprobe of claim 1, wherein said Ba and Gd have different X-ray absorption coefficients at different photon energy levels and large K-edge values to enable said bioprobe as a contrast agent for computed X-ray tomography.

6. The bioprobe of claim 1, wherein cation of said Gd (Gd³⁺) has seven unpaired inner 4f electrons exhibiting paramagnetic property which enables said bioprobe as a contrast agent for magnetic resonance imaging.

7. The bioprobe of claim 1, wherein said nanoparticles are capable of being excited at near-infrared wavelength of about 980 nm which enables said bioprobe as an upconversion fluorescent dye for fluorescence imaging and are substantially free of autofluorescence due to the upconversion luminescent property of said nanoparticles.

8. The bioprobe of claim 7 is capable of emitting green fluorescence in the cytoplasm and relatively weaker red fluorescence in the cell membrane of a target cell under the excitation of near-infrared at about 980 nm.

9. The bioprobe of claim 1, wherein said nanoparticles are capable of deep penetrating into target cell or tissue due to said surface modification on said nanoparticles.

10. The bioprobe of claim 5, wherein said Ba has X-ray absorption coefficients of about 8.51 cm² g⁻¹ and 3.96 cm² g⁻¹ at the photon energy levels of 60 keV and 80 keV respectively, and said Gd has X-ray absorption coefficients of about 1.18 cm² g⁻¹ and 5.57 cm² g⁻¹ at the photon energy levels of 60 keV and 80 keV respectively.

11. The bioprobe of claim 5, wherein said Ba has K-edge value of 37.4 keV and said Gd has K-edge value of 50.2 keV.

12. The bioprobe of claim 6, wherein each of said nanoparticles has a magnetic moment from 0.95 to 1.05 emu/g and a mass susceptibility from 4.72×10⁻⁵ to 5.2×10⁻⁵ emu/gOe at an applied magnetic field from −20 kOe to 20 kOe under room temperature.

13. The bioprobe of claim 6, wherein each of said nanoparticles has an ionic longitudinal relaxivity of about 1.194 S⁻¹ mM⁻¹.

14-27. (canceled)

28. The bioprobe of claim 1, wherein each of said nanoparticles has an average particle size of about 10 nm.

29. The bioprobe of claim 1, wherein the one-pot synthesis is a one-pot hydrothermal synthesis by using an autoclave.

30. A water soluble, single-phase and non-hydrophobic bioprobe for multi-modal bioimaging of fluorescence, magnetic resonance imaging (MRI) and computed X-ray tomography (CT) based on a plurality of nanoparticles with upconversion luminescent property, said nanoparticles comprising barium (Ba), gadolinium (Gd), fluorine (F), ytterbium (Yb), and erbium (Er);
wherein the surface of said nanoparticles is modified by poly(ethylene glycol) (PEG) moiety during a one-pot synthesis of said nanoparticles; and
wherein the nanoparticles are hydrophilic.

31. The bioprobe of claim 30, wherein each of said nanoparticles comprises a host lattice formed by Ba, Gd and F with a chemical formula of BaGdF₅ which is co-doped with Yb and Er.

32. The bioprobe of claim 31, wherein said host matrix has a face-centered cubic (FCC) phase structure and an inter-plane distance (d-spacing) of about 2.1 Å.

33. The bioprobe of claim 30, wherein each of said nanoparticles has an average particle size of about 12.02±1.55 nm.

34. The bioprobe of claim 30, wherein the one-pot synthesis is a one-pot hydrothermal synthesis by using an autoclave.

35. A water soluble, single-phase and non-hydrophobic bioprobe for multi-modal bioimaging of fluorescence, magnetic resonance imaging (MRI) and computed X-ray tomography (CT) based on a plurality of nanoparticles with upconversion luminescent property, said nanoparticles comprising barium (Ba), gadolinium (Gd), fluorine (F), ytterbium (Yb), and erbium (Er);
wherein the surface of said nanoparticles is modified by 3-mercaptopropionic acid, 6-aminocaproic acid, or a mixture thereof during a one-pot hydrothermal synthesis of said nanoparticles; and
wherein the nanoparticles are hydrophilic.

36. The bioprobe of claim 35, wherein each of said nanoparticles comprises a host lattice formed by Ba, Gd and F with a chemical formula of BaGdF₅ which is co-doped with Yb and Er.
